(19) United States
(12) Patent Application Publication
(10) Pub. No.: US 2024/0358404 A1
(43) Pub. Date: Oct. 31, 2024

(54) DISPOSABLE FURLOW INSERTION DEVICE

(71) Applicants: Boston Scientific Scimed, Inc., Maple Grove, MN (US); Boston Scientific Medical Device Limited, Galway (IE)

(72) Inventors: Amit Kumar, Noida (IN); Nitin Sharma, Gurugram (IN); Sushil Kumar, Muzaffarpur (IN); Sanjay Sundermurthy, Gurugram (IN); Arun Adhikarath Balan, Aluva (IN); Jonathan J. Lund, Glencoe, MN (US); Peeyush Tomar, Muzaffarnagar (IN)

(21) Appl. No.: 18/643,576

(22) Filed: Apr. 23, 2024

Related U.S. Application Data
(60) Provisional application No. 63/498,926, filed on Apr. 28, 2023.

(51) Int. Cl. A61B 17/34; A61F 2/26

(52) U.S. Cl. CPC A61B 17/3468; A61B 17/3496; A61F 2/26

(57) ABSTRACT

According to a general aspect, an insertion device includes an elongate member defining a lumen extending from a first end of the elongate member to a second end of the elongate member; a coupler configured to be coupled to a first end portion of the elongate member, the coupler defining a lumen; and an obturator having a first end portion, the first end portion of the obturator being configured to be disposed within the lumen defined by the elongate member and move from a first position within the lumen defined by the elongate member to a second position within the lumen defined by the elongate member.

### CROSS-REFERENCE TO RELATED APPLICATION

This application claims priority to U.S. Provisional Patent Application No. 63/498,926, filed on Apr. 28, 2023, entitled “DISPOSABLE FURLOW INSERTION DEVICE”, the disclosure of which is incorporated by reference herein in its entirety.

### TECHNICAL FIELD

This disclosure relates generally to devices for insertion of bodily implants and, more specifically, to disposable insertion devices for bodily implants, such as penile prostheses.

### BACKGROUND

One treatment for chronic, organic, male erectile dysfunction is the implantation of a penile prosthesis that mechanically erects the penis. Such prostheses can have different physical constructions (e.g., materials, mechanical structure and function, etc.) and configurations (e.g., physical dimensions, etc.). For instance, such a penile prosthesis can be in the form of a cylinder (e.g., malleable, inflatable, etc.) that is selected based on the physical requirements of an intended recipient (a patient). For instance, an appropriate penile prosthesis can be implanted within a corpus cavernosum (corpus cavernosa) of a patient's penis by medical personnel that are trained and knowledgeable regarding the implantation and use of penile prostheses.

Insertion devices (e.g., Furlow insertion devices) can be used to facilitate the insertion of a penile prosthesis into the corpus cavernosum of a patient. Such Furlow insertion devices can be used for measurement of an insertion length within the corpus cavernosum, as well as for facilitating insertion of a penile prosthesis by deploying a suture-carrying needle, where the needle and/or suture are used to introduce (draw, pull, insert, implant, etc.) the prosthesis into the corpus cavernosum.

### SUMMARY

According to a general aspect, an insertion device includes an elongate member defining a lumen extending from a first end of the elongate member to a second end of the elongate member; a coupler configured to be coupled to a first end portion of the elongate member, the coupler defining a lumen; and an obturator having a first end portion, the first end portion of the obturator being configured to be disposed within the lumen defined by the elongate member and move from a first position within the lumen defined by the elongate member to a second position within the lumen defined by the elongate member.

In some implementations, the obturator has an end, the end defines a receiving portion. In some implementations, the obturator has an end, the end of the obturator defines a receiving portion configured to receive a portion of a needle. In some implementations, the obturator has an end, the end of the obturator defines a recessed portion. In some implementations, the obturator has an end, the end of the obturator defines a conical shaped recessed portion. In some implementations, the has a first end and a second end opposite the first end, the first end defines a recessed portion, the second end includes a handle portion.

In some implementations, the obturator includes an engagement member, the engagement member being configured to engage the coupler to help prevent removal of the obturator from the lumen defined by the coupler. In some implementations, the coupler includes an engagement member configured to engage the elongate member to couple the coupler to the elongate member. In some implementations, the lumen of the coupler is configured to receive a portion of the elongate member, the coupler includes an engagement member that extends into the lumen defined by the coupler, the engagement member is configured to engage the elongate member to couple to the coupler to the elongate member.

In some implementations, the coupler includes an engagement member configured to engage the obturator. In some embodiments, the lumen of the coupler is configured to receive a portion of the obturator, the coupler includes an engagement member that extends into the lumen defined by the coupler, the engagement member is configured to engage the obturator. In some implementations, the coupler includes a first engagement member and a second engagement member, the first engagement member being configured to engage the elongate member to help couple the coupler to the elongate member, the second engagement member being configured to engage the obturator. In some implementations, the coupler includes a first engagement member extending into the lumen defined by the coupler and a second engagement member extending into the lumen defined by the coupler, the first engagement member being configured to engage the elongate member to help couple the coupler to the elongate member, the second engagement member being configured to engage the obturator.

In some implementations, the obturator includes a tapered portion. In some implementations, the elongate member defines a slot.

According to another aspect, a kit includes a bodily implant; a delivery needle configured to be coupled to the bodily implant; and a delivery device having an elongate member defining a lumen extending from a first end of the elongate member to a second end of the elongate member, a coupler configured to be coupled to a first end portion of the elongate member, and an obturator having a first end portion, the first end portion of the obturator being configured to be disposed within the lumen defined by the elongate member and move from a first position within the lumen defined by the elongate member to a second position within the lumen defined by the elongate member.

In some implementations, the elongate member defines a slot configured to receive at least a portion of the delivery needle.

In some implementations, the obturator includes a distal end portion configured to engage a portion of the delivery needle. In some implementations, the obturator has an end, the end of the obturator defines a recessed portion, the recessed portion begin configured to receive at least a portion of the delivery needle. In some implementations, the obturator has an end, the end of the obturator defines a conical shaped recessed portion, the conical shaped recessed portion being configured to receive at least a portion of the delivery needle.

### BRIEF DESCRIPTION OF THE DRAWINGS

FIG. 1 schematically illustrates a system according to an implementation.

FIG. 2 is a perspective view of an insertion device according to an implementation.

FIGS. 3 and 4 are cross-sectional views of the insertion device of FIG. 2.

FIGS. 5 and 6 are cross sectional views of a portion of the insertion device of FIG. 2.

FIG. 7 is a perspective view of an elongate member of the insertion device of FIG. 2.

FIG. 8 is a cross-sectional view of the elongate member of FIG. 7.

FIG. 9 is a side view of a coupler of the insertion device of FIG. 2.

FIG. 10 is a cross-sectional view of the coupler of FIG. 9.

FIG. 11 is side view of an obturator of the insertion device of FIG. 2.

FIG. 12 is a side view of a portion of the obturator of FIG. 11.

FIG. 13 is a side view of an obturator according to another implementation.

FIG. 14 is a side view of a portion of the obturator of FIG. 13.

FIGS. 15 and 16 are cross-sectional views of the obturator of FIG. 13.

FIG. 17-19 illustrate a method of using an insertion device according to an implementation.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as insertion devices for penile prostheses or other bodily implants. The term patient may hereafter be used for a person who benefits from the medical device or the methods disclosed in the present disclosure. For example, the patient can be a person whose body is implanted using the medical device or benefits from the methods disclosed for operating the medical devices of the present disclosure. For example, in some embodiments, the patient may be a human male, a human female, or any other mammal.

The terms proximal and distal described in relation to various devices, apparatuses, and components as discussed in the subsequent text of the present disclosure are referred to using a point of reference. The point of reference, as used in this description, is a perspective of a person who uses the disclosed insertion devices to implant a bodily implant, such as a penile prosthesis. The person may be a surgeon, a physician, a nurse, a doctor, a technician, and the like who may perform the implantation procedure. The term proximal refers to an area or portion that is closer or closest to the person during the implantation procedure. The term distal refers to an area or portion that is farther from, or farthest from the person.

The embodiments discussed herein may provide improvements to penile prosthesis insertion devices (e.g., Furlow insertion devices, Furlow insertion tools, insertion tools, devices, tools, etc.). For example, such insertion devices can be configured to be used in a single procedure and then discarded or disposed of. Some such insertion devices may have components that are coupled together and that when removed from each other would destroy the insertion device.

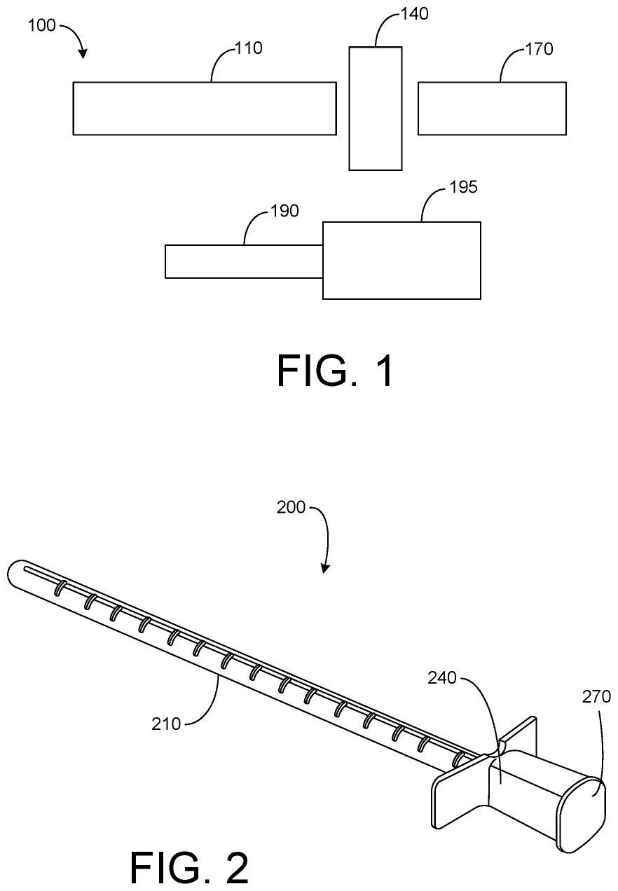

FIG. 1 schematically illustrates a system according to an implementation. The system includes a delivery or insertion device 100, an implant 195, and a needle 190 coupled to the implant 195. In some implementations, the implant 195 may be a penile implant or another type of implant that is configured to be placed in the pelvic region of the patient. In other implementations, the implant 195 is configured to be placed within the body of the patient at a different location. In some implementations, the needle 190 is coupled to the implant 195 via a suture. In other implementations, the needle 190 is directly coupled to the implant 195 or is coupled to the implant 195 via another mechanism.

The insertion device 100 includes an elongate member 110, a coupler 140, and an obturator 170. The elongate member 110 is coupled to the coupler 140 and the obturator 170 is coupled to the coupler 140. In some implementations, the elongate member 110 is fixedly coupled to the coupler 140. In some implementations, the obturator 170 is moveably, such as slidably, coupled to the coupler 140. The obturator 170 has a portion that is configured to be disposed within a lumen defined by the elongate member 110.

In use, the needle 190 or a portion of the needle 190 may be placed or disposed within the lumen defined by the elongate member. The delivery device 100 including the needle 190 may be placed within the body of the patient to a desired location. The obturator 170 may then be advanced or moved distally within the lumen defined by the elongate member 110. The obturator 170 is configured to engage the needle 190 and advance the needle 190 out of the lumen defined by the elongate member 110. The delivery device 100 may then be removed from the body of the patient and the needle can then be pulled or otherwise moved within the body of the patient until the implant 195 is disposed at a desired location.

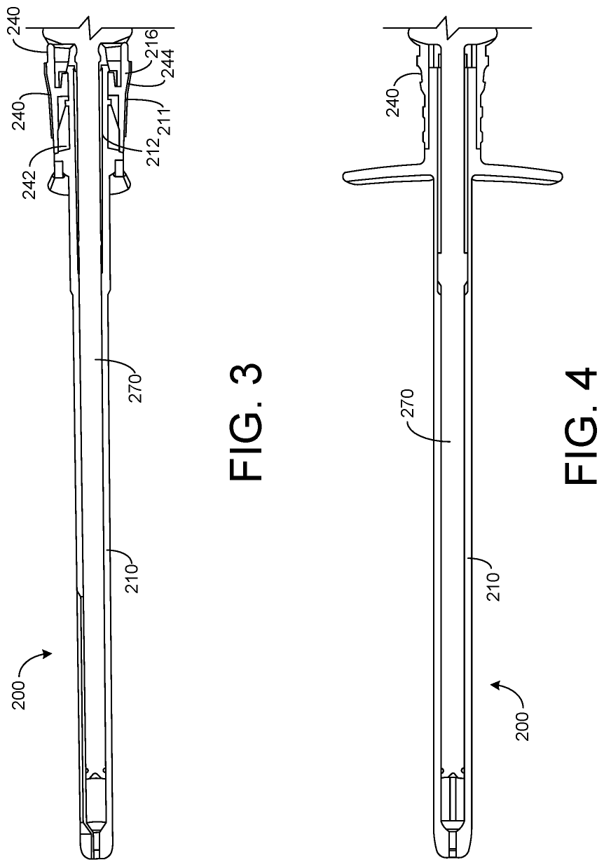

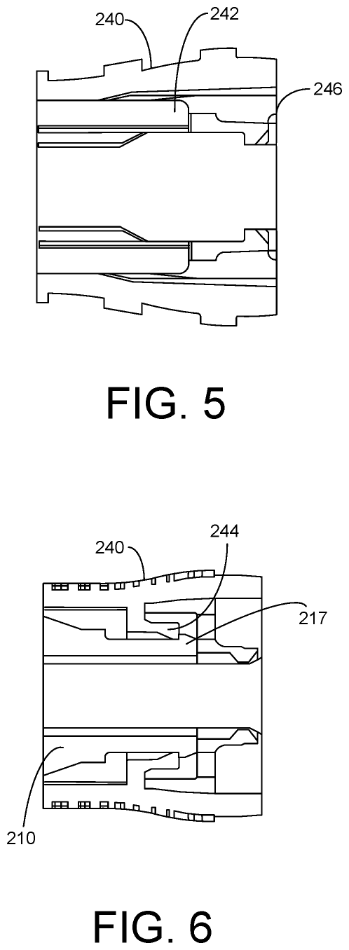

FIGS. 2-6 illustrate an insertion device 200 according to an implementation. FIG. 2 is a perspective view of the insertion device 200. FIGS. 3 and 4 are cross-sectional views of the insertion device. FIGS. 5 and 6 are cross sectional views of a portion of the insertion device.

The insertion device 200 includes an elongate member 210, a coupler 240, and an obturator 270. The elongate member 210 is coupled to the coupler 240 and the obturator 270 is coupled to the coupler 240. In the illustrated implementation, the elongate member 210 is fixedly coupled to the coupler 240.

As best illustrated in FIG. 3-6, the coupler 240 defines a lumen 242. A proximal end portion 212 of the elongate member 210 is configured to be disposed within the lumen 242 defined by the coupler 240. The proximal end portion 212 of the elongate member 210 includes an engagement portion or engagement member 217. The engagement portion or member 217 extends from an outer surface 211 of the elongate member 210. The coupler 240 also includes an engagement member 244. The engagement member 244 of the coupler 240 extends into the lumen 242 defined by the coupler 240. The engagement member 244 of the coupler 240 is configured to engage or interact with the elongate member 210 to couple the elongate member 210 to the coupler 240. As best illustrated in FIG. 6, the engagement member 244 of the coupler 240 is configured to engage or interact with the engagement member 217 of the elongate member 210. In some implementations, the engagement member 244 of the coupler 240 is configured to bend or flex to provide a snap type coupling to the elongate member 210. In other implementations, the elongate member 210 is coupled to the coupler via a different mechanism or different type of coupling.

As best illustrated in FIGS. 3-5, in the illustrated implementation, the obturator 270 is movably coupled to the coupler 240. At least a portion of the obturator 270 is disposed within the lumen 242 defined by the coupler 240 and is configured to slide, translate, or otherwise move with respect to the coupler 240. In the illustrated implementation, the coupler 240 includes an engagement member 246. As will be discussed in more detail below, the engagement member 246 extends into the lumen 242 defined by the coupler 240 and is configured to engage or contact a portion of the obturator 270.

At least a portion of the obturator 270 extends into a lumen 216 defined by the elongate member 210. The obturator 270 is configured to slide or otherwise move within the lumen 216 defined by the elongate member 210.

In use, a needle that is coupled to a bodily implant may be placed or disposed within the lumen 216 defined by the elongate member 210. The delivery device 200 including the needle may be placed within the body of the patient to a desired location. The obturator 270 may then be advanced or moved distally within the lumen 216 defined by the elongate member 210. The obturator 270 is configured to engage the needle and advance the needle out of the lumen 216 defined by the elongate member 210. The delivery device 200 may then be removed from the body of the patient and the needle can then be pulled or otherwise moved within the body of the patient until the implant is disposed or located at a desired location.

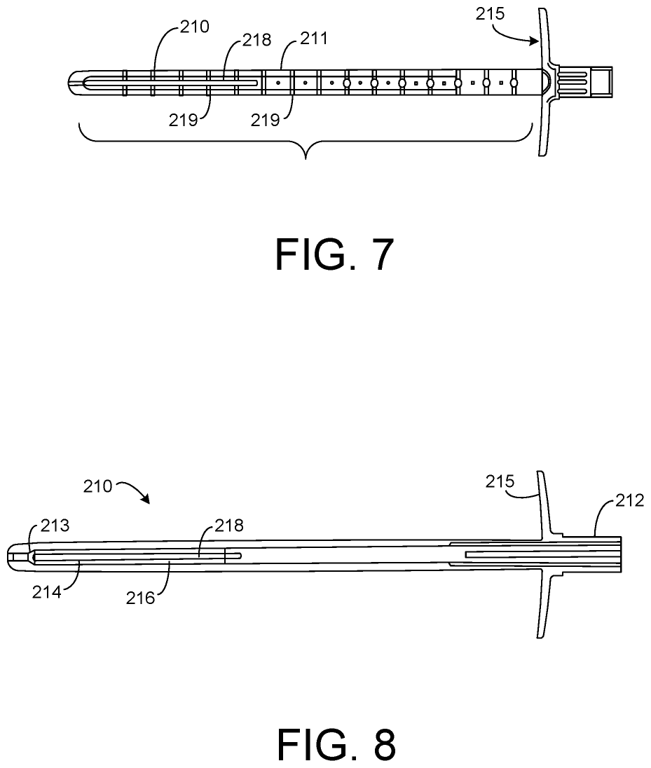

FIGS. 7 and 8 illustrate the elongate member 210. The elongate member 210 includes or defines a slot 218. The slot 218 is disposed at the distal end portion 214 of the elongate member 210 and communicates with the lumen 216 defined by the elongate member 210. The slot 218 is configured to receive the needle to facilitate the placement of the needle into the lumen 216 defined by the elongate member 210. The elongate member 210 also includes a tapering or tapered portion 213 of the lumen 216 at the distal end portion 214 of the elongate member 210. In some implementations, the tapering or narrowing of the lumen 216 (the inner diameter of the lumen gets smaller towards the end of the elongate member 210) helps eject or dispose of the needle more accurately as desired by the user. For example, the narrowing or tapering of the lumen 216 of the elongate member 210 may limit the or minimize the exit angle of the needle from the lumen 216.

In the illustrated implementation, the elongate member 210 includes a handle portion 215. The handle portion 215 extends from an outer surface 211 of the elongate member 210. The handle portion 215 may be grasped by the user during the insertion of the delivery device 200 into the body of the patient and during the advancement or movement of the obturator 270 with respect to the elongate member 210.

The elongate member 210 includes measurement markings 219. The measurement markings 219 may be configured to indicate to the user how far the elongate member 210 (or the delivery device 200) has been inserted into the body of the patient. In some implementations, the measurement markings 219 are laser etched onto the material of the elongate member 210. In such implementations, the surface of the elongate member 210 that includes the measurement markings 219 may be smooth. In other implementations, the measurement markings 219 may be formed in a different manner. For example, the measurement markings 219 may be painted, colored, or otherwise marked on the surface of the elongate member 210.

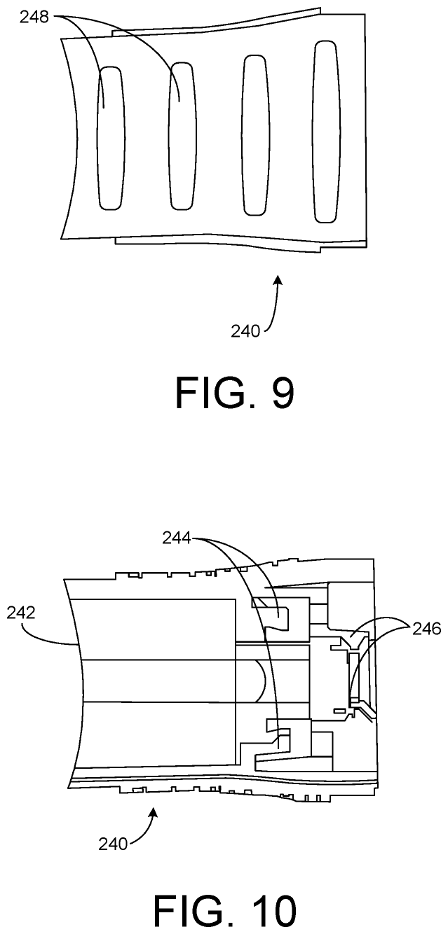

FIGS. 9 and 10 illustrate the coupler 240. In the illustrated implantation, the coupler 240 defines a lumen 242. The lumen 242 is configured to receive at least a portion of the elongate member 210 and at least a portion of the obturator 270. The coupler includes an engagement member 244 that is configured to engage the elongate member 210 to couple the elongate member 210 to the coupler 240. The engagement member 244 extends into the lumen 242 defined by the coupler 240. In some implementations, the engagement member 244 is configured to bend or flex.

The coupler includes an engagement member 246. The engagement member 246 extends into the lumen 242 defined by the coupler 240 and is configured to engage the obturator 270. In some implementations, the engagement member 246 is configured to contact or engage the obturator 270 to help prevent the obturator 270 from being removed from the coupler 240. Accordingly, the obturator 270 may move or slide within the lumen 242 defined by the coupler 240 but in some implementations, may not be removed entirely from the lumen 242 of the coupler 240.

In the illustrated implementation, the coupler 240 includes ridges or projections 248 that extend from an outer surface of the coupler 240. The ridges or projections 248 may facilitate the grasping or gripping of the coupler 240 or the delivery device 200 during use of the device.

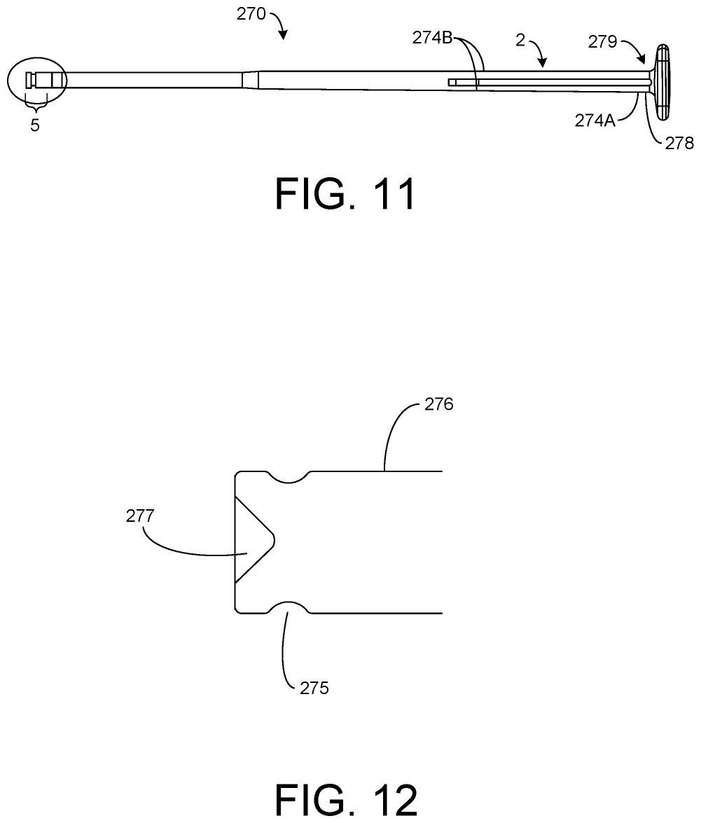

FIGS. 11 and 12 illustrate the obturator 270 of the delivery device 200. The obturator 270 includes ramps or bumps 272A and 272B. The ramps or bumps extend from an outer surface 271 of the obturator 270 and are configured to engage the coupler 240. For example, in some implementations, the ramps or bumps 272A and 272B are configured to engage the engagement member 246 of the coupler 240. For example, in the illustrated implementation, the proximal ramps 272A may engage the engagement member 246 of the coupler 240 when the obturator is placed in its distal most position. The distal ramp 272B may be configured to engage the engagement member 246 of the coupler 240 when the obturator is placed in its proximal most position. Additionally, in some implementations, the distal ramps 272B may engage the engagement member 246 of the coupler 240 to help prevent the removal of the obturator 270 from the coupler 240. In the illustrated implementation, the obturator 270 includes two proximal ramps or bumps 272A and two distal ramps or bumps 272B. In other implementations, the obturator includes a different number of ramps or bumps.

In the illustrated implementation, the obturator 270 includes an association ramp or bump 274. The association ramp or bump 274 is configured to facilitate the association of the obturator 270 and the coupler 240 and is configured to help prevent the removal of the obturator 270 from the coupler 240. For example, in some implementations, the association ramp or bump 274 is configured to contact or engage the coupler 240 to prevent the removal or disassociation of the obturator 270 from the coupler 240.

The distal end 276 of the obturator 270 includes or defines a recess 277. The recess 277 is configured to receive a portion of the needle. In some implementations, the recess 277 is configured to receive a portion of the needle so that the needle may then be advanced by the obturator 270 within the lumen of the elongate member 210 in a linear fashion (rather than pushing or advancing the needle at an angle). The linear advancement of the needle may facilitate a more accurate placement of the needle within the body of the patient. In the illustrated implementation, the recess 277 has a conical shape. In other implementations, the recess 277 has a different shape, such as rectangular or spherical.

In the illustrated implementation, the obturator 270 also includes a groove 275. The groove 275 is disposed near the distal end 276 of the obturator 270. The groove 275 allows for less surface area of the outer surface of the obturator 270 to contact the inner surface of the lumen of the elongate member 210. Accordingly, there is less friction between the surfaces to facilitate movement of the obturator 270 with respect to the elongate member 210.

The proximal end portion 278 of the obturator 270 includes a handle portion 279. The handle portion 279 may be grasped by the user to move the obturator 270 with respect to the coupler 240 and to the elongate member 210.

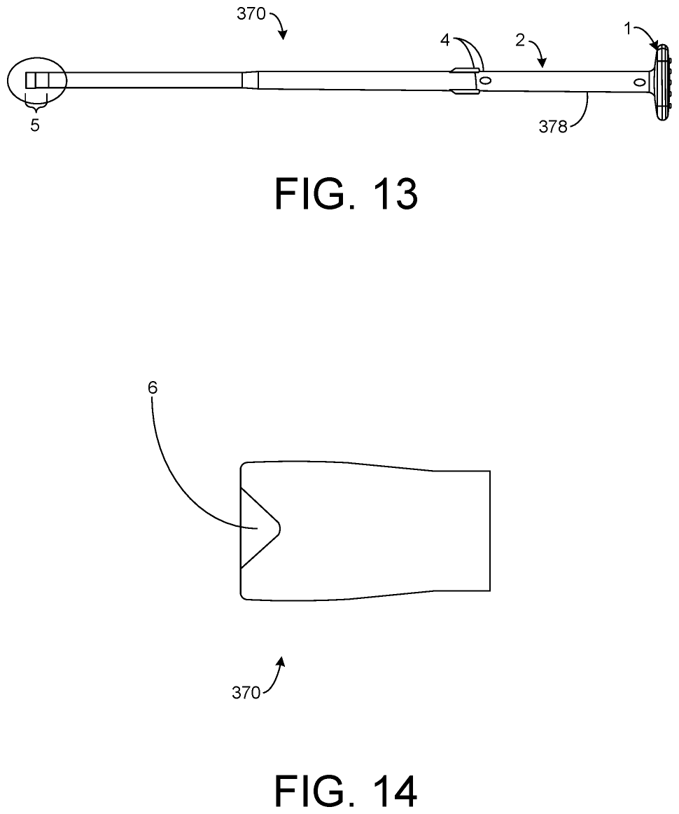

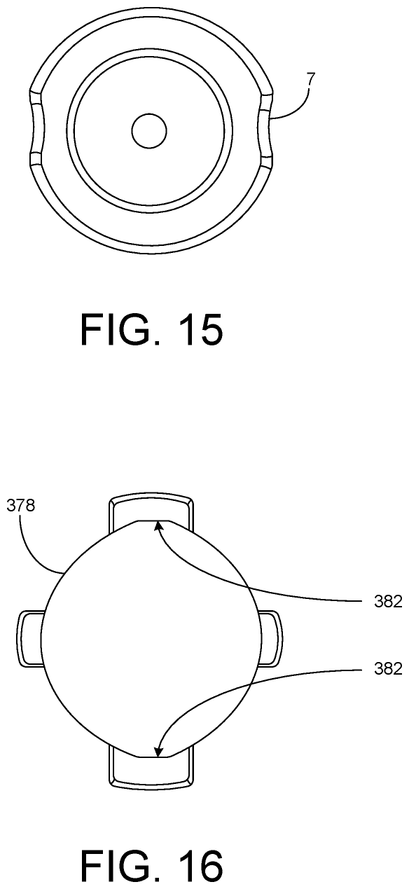

FIGS. 13 to 16 illustrate an obturator 370 according to another implementation. As best illustrated in FIG. 16, the proximal end portion 378 of the obturator 370 includes extension portions 382. The extension portions 382 extend from the surface of the obturator 370 and are configured to engage the coupler. The engagement of the extension portions 382 with the coupler provide tactile feedback to the user. Accordingly, the user may more accurately move or slide the obturator 370 with respect to the coupler.

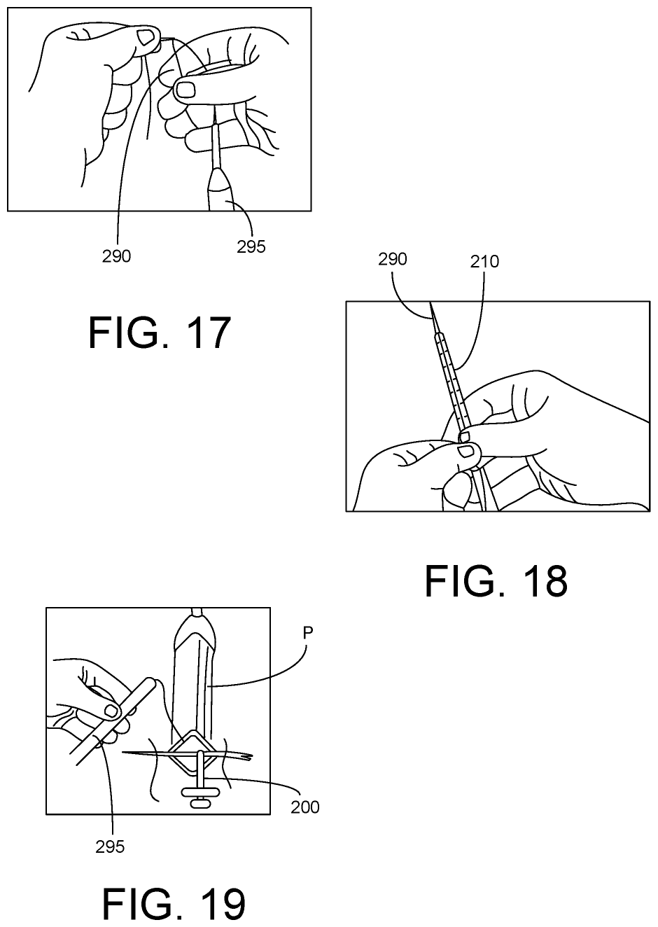

FIGS. 17 to 19 illustrate steps for placing an implant within a body of a patient using a delivery device. As illustrated in FIG. 17, a needle 290 may be associated with or coupled to an implant 195. In some cases, the needle 290 may be associated with the implant 195 via a suture. As illustrated in FIG. 18, the needle 290 may be placed within the lumen defined by the elongate member 210 of the delivery device 200. In some cases, the needle 290 may be inserted into the lumen via a slot defined by the elongate member 210. As illustrated in FIG. 19, the delivery device 200 may be inserted into the body of the patient, such as into a penis P of the patient. Once the delivery device 200 including the needle 290 are placed within the body of the patient at a desired location, the obturator 270 may then be advanced or moved distally within the lumen defined by the elongate member 210. The obturator 270 is configured to engage the needle 290 and advance the needle 290 out of the lumen defined by the elongate member 210. The delivery device 200 may then be removed from the body of the patient and the needle 290 can then be pulled or otherwise moved within the body of the patient until the implant 295 is disposed at a desired location.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An insertion device comprising:
an elongate member defining a lumen extending from a first end of the elongate member to a second end of the elongate member;
a coupler configured to be coupled to a first end portion of the elongate member, the coupler defining a lumen; and
an obturator having a first end portion, the first end portion of the obturator being configured to be disposed within the lumen defined by the elongate member and move from a first position within the lumen defined by the elongate member to a second position within the lumen defined by the elongate member.

2. The insertion device of claim 1, wherein the obturator has an end, and the end defines a receiving portion.

3. The insertion device of claim 1, wherein the obturator has an end, and the end of the obturator defines a receiving portion configured to receive a portion of a needle.

4. The insertion device of claim 1, wherein the obturator has an end, and the end of the obturator defines a recessed portion.

5. The insertion device of claim 1, wherein the obturator has an end, and the end of the obturator defines a conical shaped recessed portion.

6. The insertion device of claim 1, wherein the obturator has a first end and a second end opposite the first end, the first end defines a recessed portion, the second end includes a handle portion.

7. The insertion device of claim 1, wherein the obturator includes an engagement member, the engagement member being configured to engage the coupler to help prevent removal of the obturator from the lumen defined by the coupler.

8. The insertion device of claim 1, wherein the coupler includes an engagement member configured to engage the elongate member to couple the coupler to the elongate member.

9. The insertion device of claim 1, wherein the lumen of the coupler is configured to receive a portion of the elongate member, the coupler includes an engagement member that extends into the lumen defined by the coupler, and the engagement member is configured to engage the elongate member to couple to the coupler to the elongate member.

10. The insertion device of claim 1, wherein the coupler includes an engagement member configured to engage the obturator.

11. The insertion device of claim 1, wherein the lumen of the coupler is configured to receive a portion of the obturator, the coupler includes an engagement member that extends into the lumen defined by the coupler, and the engagement member is configured to engage the obturator.

12. The insertion device of claim 1, wherein the coupler includes a first engagement member and a second engagement member, the first engagement member being configured to engage the elongate member to help couple the coupler to the elongate member, and the second engagement member being configured to engage the obturator.

13. The insertion device of claim 1, wherein the coupler includes a first engagement member extending into the lumen defined by the coupler and a second engagement member extending into the lumen defined by the coupler, the first engagement member being configured to engage the elongate member to help couple the coupler to the elongate member, and the second engagement member being configured to engage the obturator.

14. The insertion device of claim 1, wherein the obturator includes a tapered portion.

15. The insertion device of claim 1, wherein the elongate member defines a slot.

16. A kit, comprising:
a bodily implant;
a delivery needle configured to be coupled to the bodily implant; and
a delivery device having an elongate member defining a lumen extending from a first end of the elongate member to a second end of the elongate member, a coupler configured to be coupled to a first end portion of the elongate member, and an obturator having a first end portion, the first end portion of the obturator being configured to be disposed within the lumen defined by the elongate member and move from a first position within the lumen defined by the elongate member to a second position within the lumen defined by the elongate member.

17. The kit of claim 16, wherein the elongate member defines a slot configured to receive at least a portion of the delivery needle.

18. The kit of claim 16, wherein the obturator includes a distal end portion configured to engage a portion of the delivery needle.

19. The kit of claim 16, wherein the obturator has an end, the end of the obturator defines a recessed portion, the recessed portion being configured to receive at least a portion of the delivery needle.

20. The kit of claim 16, wherein the obturator has an end, the end of the obturator defines a conical shaped recessed portion, the conical shaped recessed portion being configured to receive at least a portion of the delivery needle.
